(19) United States
(12) Patent Application Publication
(10) Pub. No.: US 2023/0270779 A1
(43) Pub. Date: Aug. 31, 2023

(54) EXOSOME HYDROGEL FOR TREATING SEVERE RESPIRATORY DISEASES AND PREPARATION METHOD THEREOF

(71) Applicants: Tangyi Holdings(Shenzhen) Limited, Shenzhen (CN); Healthina Stem Cell Industry Platform (Tianjin) Limited, Tianjin (CN)

(72) Inventors: Bin ZHENG, Tianjin (CN); Yulin CAO, Beijing (CN); Tingting HUA, Tianjin (CN); Shixiang CHENG, Tianjin (CN)

(73) Assignees: Tangyi Holdings(Shenzhen) Limited, Shenzhen (CN); Healthina Stem Cell Industry Platform (Tianjin) Limited, Tianjin (CN)

(21) Appl. No.: 17/653,465

(22) Filed: Mar. 4, 2022

(30) Foreign Application Priority Data
Jan. 26, 2022 (CN) 202210095397.1

(51) Int. Cl. A61K 35/12; A61K 9/06; A61K 47/10; A61K 47/34; A61P 11/00

(52) U.S. Cl. CPC A61K 35/12; A61K 9/06; A61K 47/10; A61K 47/34; A61P 11/00

(57) ABSTRACT

An exosome hydrogel for treating severe respiratory diseases is provided and includes a temperature-sensitive hydrogel and an exosome; wherein the temperature-sensitive hydrogel is PLGA-PEG-PLGA. According to the present invention, PLGA-PEG-PLGA generates a hydrogel exosome through a phase transition at a body temperature, and the hydrogel stays in lungs for a longer time than common exosomes, thereby making the exosome remain at an infection site and producing a better therapeutic effect. The exosome hydrogel features high safety, is suitable for promotion, and has good potential in medical treatment.

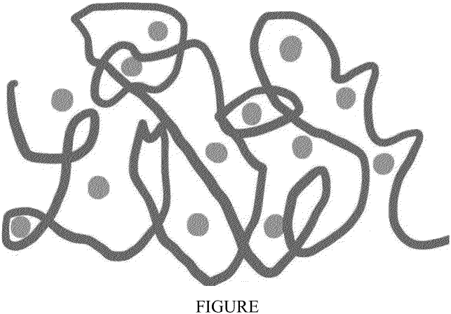

### CROSS REFERENCE TO THE RELATED APPLICATIONS

This application is based upon and claims priority to Chinese Patent Application No. 202210095397.1, filed on Jan. 26, 2022, the entire contents of which are incorporated herein by reference.

### TECHNICAL FIELD

The present invention relates to the technical field of hydrogel, in particular to hydrogel synthesis and treatment for severe respiratory diseases by a hydrogel-delivered exosome, and further in particular to an exosome hydrogel for treating severe respiratory diseases and a preparation method thereof.

### BACKGROUND

Patients with severe respiratory diseases are manifested as relatively severe pulmonary inflammation, with functions of organs other than lungs affected, such as notable acceleration in respiratory frequency, confusion and unconsciousness, unstable blood pressure and reduced urinary output, thereby affecting functions of other important organs. Severe respiratory diseases can be life threatening. At present, the mortality rate of severe respiratory diseases is 20%-60%, and in particular for middle-aged and elderly people, and infants and young children, the mortality rate reaches more than 50%. Therefore, a more effective treatment for severe respiratory diseases is in urgent need.

Exosomes are vesicle structures secreted by mesenchymal stem cells, including various regulatory factors such as cytokines, growth factors, signal lipids, messenger RNA (mRNA), and non-coding RNA (miRNA), which play an important role for mesenchymal stem cells in regulating other cells in the surrounding environment. Exosomes sourced from mesenchymal stem cells can carry drugs with therapeutic effects into target cells, and nanoscale exosomes tend to reach damaged tissue regions after being injected and have advantages of low immunogenicity and tumorigenicity.

### SUMMARY

The present invention is intended to provide an exosome hydrogel for treating severe respiratory diseases and a preparation method thereof. The exosome hydrogel achieves a better therapeutic effect for severe respiratory diseases by extending the action time of an exosome in lungs, features high safety, is suitable for promotion, and has good potential in medical treatment.

To achieve the above-mentioned purpose, the present invention provides an exosome hydrogel for treating severe respiratory diseases, including a temperature-sensitive hydrogel and an exosome;

wherein the temperature-sensitive hydrogel is poly (lactic-co-glycolic acid)-polyethylene glycol-poly (lactic-co-glycolic acid) (PLGA-PEG-PLGA).

An exosome hydrogel for treating severe respiratory diseases and a method for treating severe respiratory diseases using the exosome hydrogel include the following steps:

mixing PLGA-PEG-PLGA with an exosome, and delivering the mixture into a nasal cavity, a respiratory tract and lungs, wherein PLGA-PEG-PLGA generates a hydrogel through a phase transition at a body temperature due to a temperature change.

Therefore, according to the exosome hydrogel for treating severe respiratory diseases and the method for treating severe respiratory diseases using the exosome hydrogel of the present invention, PLGA-PEG-PLGA remains liquid at a room temperature; after entering a nasal cavity, a respiratory tract and lungs, PLGA-PEG-PLGA generates a hydrogel exosome through a phase transition at a body temperature due to a temperature change, and the hydrogel stays in lungs for a longer time than common exosomes, thereby making the exosome remain at an infection site and producing a better therapeutic effect. The exosome hydrogel features high safety, is suitable for promotion, and has good potential in medical treatment.

Specific technical benefits of the present invention are as follows:

(1) The exosome hydrogel of the present invention includes two parts, i.e., a temperature-sensitive hydrogel and an exosome. The exosome hydrogel stays in lungs for a longer time than common exosomes, and achieves a better therapeutic effect for the severe respiratory diseases by extending the action time of the exosome in lungs through making full use of an adhesive capacity of the hydrogel and therapeutic effects of the exosome.

(2) The exosome hydrogel of the present invention is easy to prepare and operate, can be modularized and extended, has no high requirements on technology, is safe and suitable for promotion.

(3) The exosome hydrogel of the present invention can be inoculated by snorting, thereby avoiding the problems of great economic pressure, poor compliance and pain caused by injection when injection drugs are used, and reducing the biological hazard of needles/syringes and the risk of disease transmission.

The technical solution of the present invention will be further described below in combination with the accompanying drawings and the embodiments of the present invention.

### BRIEF DESCRIPTION OF THE DRAWINGS

The FIGURE is a schematic diagram of an exosome hydrogel of the present invention.

### DETAILED DESCRIPTION OF THE EMBODIMENTS

The technical solution of the present invention is further described below in combination with the accompanying drawings and the embodiments.

Unless otherwise defined, the technical terms or scientific terms used in the present invention should have the ordinary meaning as understood by those of ordinary skills in the art.

### Test

### (I) Materials and Instruments

PLGA-PEG-PLGA: purchased from Guangzhou Tansh Technology Co., Ltd.;
Exosome reagent extraction kit: purchased from Umibio (Shanghai) Co., Ltd.;
Stem cells: purchased from Shanghai Jinyuan Biotechnology Co., Ltd.;
Serum-containing mesenchymal stem cell medium: purchased from Shanghai Zhongqiao Xinzhou Biotechnology Co., Ltd.

### (II) Preparation of Exosome

### S1. Sample Pretreatment

Cell debris was centrifuged and removed: a sample was transferred into a centrifuge tube and centrifuged at 4° C. for 10 min with a separation factor of 3,000 g, then cell debris in cell supernatant was removed, and the centrifuged cell supernatant without cell debris was transferred into a new 50 mL centrifuge tube.

### S2. Extraction of Exosome

5 ml of ECS was added into 20 mL of cell supernatant, the centrifuge tube was tightly covered, and such solution was mixed well in a vortex oscillator for 1 min and then kept static at 4° C. for 2 h. The centrifuge tube with the mixture was centrifuged at 4° C. for 60 min with a separation factor of 10,000 g, then the cell supernatant was discarded, 1×PBS was taken to evenly blow up centrifuged precipitation, and after the precipitation was dissolved, a resuspension solution was transferred to a new 1.5 mL centrifuge tube. The 1.5 mL centrifuge tube with the resuspension solution was centrifuged at 4° C. for 2 min with a separation factor of 12,000 g, and the supernatant was retained, which was rich in exosome particles.

S3. Purification of the exosome. The resulting crude exosome particles were transferred into an EPF column upper chamber and centrifuged at 4° C. for 10 min with a separation factor of 3,000 g, and after centrifugation, liquid at a bottom of the EPF column was collected, which was the purified exosome particles.

### (III) Preparation of Exosome Hydrogel Examples PS0-PS2

The preparation method included the following steps: 20 mg of temperature-sensitive hydrogel PLGA-PEG-PLGA and 1 mg of exosome were mixed well.

TABLE 1

Exosome hydrogel examples PS0-PS2

| Group | Exosome hydrogel preparation examples | | |
|---|---|---|---|
| | PS0 | PS1 | PS2 |
| Exosome | No | Yes | Yes |
| PLGA-PEG-PLGA | Yes | No | Yes |

### (IV) Treatment for Severe Respiratory Diseases by the Exosome Hydrogel

Exosome hydrogel drugs were prepared by different components, and a recovery rate of severe respiratory diseases was tested with the different exosome hydrogel drugs. The test results are shown in Table 2.

TABLE 2

Recovery rates of severe respiratory diseases treated by control samples and exosome hydrogel examples

| Group | Exosome hydrogel preparation examples | | |
|---|---|---|---|
| | PS0 | PS1 | PS2 |
| Exosome | No | Yes | Yes |
| PLGA-PEG-PLGA | Yes | No | Yes |
| Recovery rate of severe respiratory diseases/% | 8 | 59 | 75 |

As shown in Table 2, PS2 has the highest recovery rate of severe respiratory diseases and a better therapeutic effect for severe respiratory diseases.

Therefore, according to the exosome hydrogel for treating severe respiratory diseases and the method for treating severe respiratory diseases using the exosome hydrogel of the present invention, the exosome remains at an infection site and produces a better therapeutic effect. The exosome hydrogel features high safety, is suitable for promotion, and has good potential in medical treatment.

It should be noted that the above-mentioned embodiments only serve to illustrate the technical solution of the present invention rather than to restrict it. Though the present invention is described in detail with reference to preferred embodiments, those of ordinary skills in the art should understand that they can make modification or equivalent replacement to the technical solution of the present invention, and such modification or equivalent replacement should not make the modified technical solution depart from the spirit and scope of the technical solution of the present invention.

What is claimed is:

1. An exosome hydrogel for treating severe respiratory diseases, comprising a temperature-sensitive hydrogel and an exosome;
wherein the temperature-sensitive hydrogel is a poly (lactic-co-glycolic acid)-polyethylene glycol-poly (lactic-co-glycolic acid) (PLGA-PEG-PLGA).

2. A method for treating severe respiratory diseases using the exosome hydrogel according to claim 1, comprising the following steps:
mixing the PLGA-PEG-PLGA with the exosome, and delivering a mixture of the PLGA-PEG-PLGA and the exosome into a nasal cavity, a respiratory tract and a lung, wherein the PLGA-PEG-PLGA generates the temperature-sensitive hydrogel through a phase transition at a body temperature due to a temperature change.
